(19) United States
(12) Patent Application Publication
(10) Pub. No.: US 2008/0241216 A1
(43) Pub. Date: Oct. 2, 2008

(54) Preparation Containing Active and/or Auxiliary Substances, With Controllable Release of Said Substances, As Well As Its Use and Manufacture

(75) Inventors: Christian VON FALKENHAUSEN, Meckenheim (DE); Markus KRUMME, Neuwied (DE)

Correspondence Address: BIRCH STEWART KOLASCH & BIRCH, PO BOX 747, FALLS CHURCH, VA, 22040-0747, US

(73) Assignee: LTS Lohmann Therapie-Systeme AG, Andernach (DE)

(21) Appl. No.: 12/031,570

(22) Filed: Feb. 14, 2008

Related U.S. Application Data
(63) Continuation of application No. 10/089,444, filed on May 22, 2002, now abandoned.

(30) Foreign Application Priority Data
Sep. 30, 1999 (DE) 199 46 822.2
Sep. 16, 2000 (EP) PCT/EP00/09061

(51) Int. Cl. A61K 9/10; A61K 9/70; A61F 2/00; A61K 9/02

(52) U.S. Cl. 424/426; 424/484; 424/448; 424/436; 424/433

(57) ABSTRACT

A preparation containing active and/or auxiliary substance(s) for the time- and/or dose-controllable release of said substances, comprising at least two layers (1, 2) in rolled or folded shape, is characterized in that a) the first layer contains at least one active or auxiliary substance, is continuous at least in sections thereof, that at least one of the parameters thickness, width and concentration of the active and/or auxiliary substance of this layer is not constant, and b) in that the second layer is continuous and possesses a lower moisture permeability than the first layer.

### RELATED APPLICATION INFORMATION

This application is a Continuation of co-pending U.S. application Ser. No. 10/089,444, filed Mar. 29, 2000, which in turn is the National Stage under 35 U.S.C. §371 of International Application PCT/EP00/09061, having an international filing date of Sep. 16, 2000 and which designated the United States of America. This application also claims priority under 35 U.S.C. §119(a-d) of German application 199 46 822.2, filed Sep. 30, 1999. The entire contents of all of the above applications are hereby incorporated in their entirety by reference.

### BACKGROUND OF THE INVENTION

1. Field of the Invention

This invention relates to preparations containing active and/or auxiliary substances, for time- and/or dose-controllable release of said substances, said preparations containing at least two layers in rolled or folded form.

2. Description of the Related Art

Active substance-containing preparations of whatever administration form generally release the active substance by diffusion or disintegration, which as a rule results in non-linear release kinetics. Embodiments of such systems can be applied as oral, rectal or vaginal administration forms, or if required also as implants. Here, a demand frequently placed on the application form is the linear release of active substance from the preparation. However, it may also be desirable to freely modulate the release profile in correspondence with the specific demands placed on a therapeutic form. Preparations for such controlled, for example linear, active substance release are mostly of a complicated structure and are expensive in manufacture.

From the state of the art are known a number of active- and auxiliary substance-containing preparations, in particular with retarded release of the ingredients. DE 43 41 442 describes an oral administration form consisting of a central, active substance-containing, non-erodible layer and a further, largely active substance-free, erodible layer enveloping said layer. Active substance release takes place by passive diffusion from the central layer, the latter being exposed to the release medium with a defined area. The reduction in the amount of active substance released per unit time is compensated by the active substance that is additionally released from the coat layer as a consequence of erosion. The principle of providing new, “undepleted” surfaces by means of erosion of largely active substance-free cover layers enables extensive modulation of the release kinetics by means of targeted selection of core and coat layer geometries. The core of the invention of the aforementioned documents thus comprises the successive provision of new surfaces of active substance-containing layers.

U.S. Pat. No. 3,625,214 describes a planar, helical, rolled-up administration form comprising two layers; the outward-facing layer being an active substance-free film which is soluble in water but is impermeable and which is coated with a water-soluble and active substance-containing matrix which is rolled inwardly, and said matrix possibly possessing a thickness profile along its extension. When this administration form is exposed to a body fluid, the outer layer erodes or dissolves and consequently exposes active substance-containing matrix material. This dissolves in the body fluid and thereby releases active agent. As a consequence of the helical winding, internal areas are exposed with delay which results in a retarded release of active substance, which release, due to varying thicknesses of the active substance-containing matrix, may have dose-modulated characteristics. Thus, the control of active substance release is accomplished here in terms of time by the exposure of new surfaces, and in terms of the dose by different thicknesses of the active agent-carrying layer.

DE 197 15 794 C1 describes a laminar drug form and a process for its manufacture. The invention for controlled active agent release comprises helically rolled-up, or folded layers on a polymer film which contains a pharmaceutically active agent. The invention is characterized in that the outer surface of the active substance-containing polymer film, which surface is accessible to the digestive juices, in the rolled-up or folded state accounts for at most 25% of its total surface area, and the rolled-up or folded layers stick to one another such that in the release test according to USP 23, Method A, Apparatus 2, at 37° C. and 50 rpm, the laminar medicament form retains its spirally coiled or folded shape in synthetic gastric juice for at least one hour, and at least 30% of the contained active substance is released in the rolled-up or folded state.

U.S. Pat. No. 4,767,627 describes an active substance delivery preparation with extended retention time in the stomach comprising a planar figure of an erodible polymer which releases an active substance contained therein over a controlled, predictable and extended period of time.

U.S. Pat. No. 4,268,497 describes a preparation for oral administration in veterinary medicine containing a medicament in an erodible film. Said film has a first shape enabling oral administration, and a second shape in the stomach, causing its retention.

### BRIEF DESCRIPTION OF THE DRAWING FIGURES

FIGS. 1a-1b show a side view of the preparation according to embodiments of the invention;

FIGS. 2a-2g show views of partially unrolled preparations according to embodiments of the invention;

FIG. 3 shows an active substance layer being formed by two different regions according to an embodiment of the invention;

FIG. 4 shows a preparation having two regions according to an embodiment of the invention;

FIGS. 5a-5b show plan and side views of layers that may contain or be free of active agent, and may be soluble or insoluble in water according to an embodiment of the invention; and

FIGS. 6a-6b show plan and side views of a preparation containing an active agent according to an embodiment of the invention.

### DETAILED DESCRIPTION

Starting from the aforementioned state of the art, it is the object of the present invention to provide an application form for an active substance-containing preparation which is less complicated in manufacture and enables a freely modulatable release with simple as well as inexpensive means.

To achieve this object it is proposed according to the present invention, for a preparation possessing the features mentioned in the introductory part of the main claim to contain at least one active or auxiliary substance in the first layer, and that said layer is continuous at least in sections thereof, and that at least one of the parameters thickness, width and concentration of the active or auxiliary substance of this layer is not constant. In addition, the preparations according to the present invention are characterized in that the second layer is continuous and possesses a lower moisture permeability than the first layer.

In accordance with the above, the invention relates to a preparation containing active and/or auxiliary substances which has the aforementioned features, for time- and/or dose-controlled release of said substances, said preparation containing at least two layers (1, 2) in rolled-up or folded form.

The carrier layer (1) may either be covered along its entire length by an active agent-free matrix layer (2), but it may also have active agent-containing regions in longitudinal direction such that the active agent-containing and active agent-free regions alternate at distances. Furthermore, the carrier layer may in its longitudinal direction also possess regions with matrix layers containing different active and/or auxiliary substances.

Especially advantageous are such embodiments which have at least one continuous and largely moisture-impermeable layer. This layer too, may if it appears advantageous, contain active substances or auxiliary substances, or both at the same time.

Via this moisture-impermeable layer the diffusion of water or body fluids—and the degradation of the layer by erosion, dissolution, etc., associated therewith—takes place at a slower pace than is the case in the active substance-containing layers, so that in the latter the degradation of the layer and thereby the release of active substance starts earlier.

A further embodiment provides for at least one of the layers (2) of the laminate to be soluble or erodible in body fluid, and for another layer (1) to be less soluble or more difficult to erode, or even insoluble or non-erodible.

It is to be noted here that there exists an interaction between solubility or degradability on the one hand and the thickness of the material on the other. Thus, a largely insoluble material may be configured comparatively thin, while on the other hand in the case of moisture-permeable, more readily soluble, erodible or biodegradable materials, the layers must be of a correspondingly greater thickness.

Usually, the active agent concentration is the same everywhere along the longitudinal extension of the active agent-containing layer. However, it may be of advantage for the concentration of the active substance or active substances to be different in relation to the longitudinal extension of the active substance-containing layer(s), as according to a preferred embodiment of the invention. In this case, the differences in concentration may preferably be configured in the form of a concentration gradient, or in the form of an otherwise variable concentration profile.

In addition, one may make use of the feature of at least one layer being pressure-sensitive adhesive. The active substance layer, also called matrix, may over its entire length have uniform thickness; in this case the width of said layer may vary along its extension in longitudinal direction. The result of this is the so-called width profile.

In one embodiment of the invention, in the case of a rolled-up laminate, the outer layer may be active agent and/or auxiliary agent-containing.

However, in another embodiment, it is also possible for a pressure-sensitive adhesive, liquid-soluble active substance layer to be provided on the inside of the winding so that thereby the largely active agent-free carrier layer prevents a premature release of active agent. When this spirally wound up preparation is exposed to a body fluid, the active substance-containing adhesive dissolves and partially unrolls the system. In accordance with the surface area that has been exposed at any given moment, active substance can then enter from the said layer into the body fluid by diffusion or solution. Thus, the release profile is controlled by the geometry of the active substance layer. In this process, the slow unrolling of the system successively exposes new active substance-containing surfaces, so that the release profile results from the layer geometry and the speed of unrolling.

In a further embodiment of the invention, provision may be made for the measure of arranging the active substance-containing layer on the outside of the spiral, whereas the inner winding is formed by the carrier layer.

An advantage of this embodiment is the initial dose provided by the active substance-containing outer winding.

A further embodiment of the invention provides for layer regions with active and/or auxiliary agents to be present which differ in terms of their ingredients and/or their solubility, adhesive power or erosion properties.

As a consequence, the release profile can additionally be further modulated, and, in particular, can be imprinted in a dose-modulated manner in the process. The control of active substance release in this embodiment is accomplished in chronologically successive “pulses” by exposure of different surfaces comprising different active and auxiliary substances.

The invention thereby enables the release of different active agents with differing active substance kinetics. For example, the active substance layer can be formed by two regions carrying different active substances, one of said regions providing pulsed release and the other region enabling a continuous release of active substance.

The invention further comprises the possibility of winding the laminate, which is present in sheet-like form, on a winding core, which is removed after completion of the winding, so that a central recess results. This recess may be 0.5 to 30 mm in diameter, preferably 1 to 10 mm, more preferably 2 to 5 mm.

Furthermore, the winding core may also remain in the system as a component of the preparation; said winding core may be compact or hollow, i.e. configured as a ring, contain an active substance or be configured to be largely free of active substance. In addition, the width of the winding core may exceed the maximum width of the laminate. The diameter of said winding core is 0.5 mm to 30 mm, preferably 1 to 10 mm, and more preferably 2 to 5 mm.

The active substance release may be effected by diffusion and/or dissolution of the active substance from an active substance layer which is largely insoluble in acid and/or basic environment, or by degradation or dissolution of an active substance layer which is soluble in acid and/or basic environment.

To produce a preset active substance release profile, it may be advantageous for the thickness of a layer to be in the range of between 1 μm and 500 μm, preferably between 5 μm and 150 μm, more preferably between 10 μm and 30 μm. The width of an active agent-containing layer may be in the range between 1 mm and 50 mm, preferably between 1 mm and 30 mm, more preferably between 10 mm and 30 mm.

It may in addition be of advantage for the purposes of the present invention that the area of the active substance layer, relative to the carrier layer, be in the region of between 1 and 99%, preferably between 10 and 80%, more preferably between 30 and 70%. The unwound length of the total system may advantageously be in the range of between 5 mm and 300 mm, preferably between 10 mm and 200 mm, more preferably between 10 mm and 50 mm.

With respect to the release profile, such embodiments of the invention are particularly preferred as are characterized by a linear course of release. Furthermore, those embodiments are especially preferred which have the capability of releasing an initial dose. The initial dose may be provided, for instance, by means of an active substance-containing outer winding.

It may also be of advantage if the rolled-up or folded preparations of the invention are provided with additional cover layers at those sides which correspond to the longitudinal sides of the respective layers. This creates a protection against the attack of water or body fluids. Preferably, said lateral cover layers comprise largely moisture-impermeable materials.

For the manufacture of suitable administration forms, the rolled-up or folded preparations of the invention are preferably imbedded in a substrate which may consist of a substance soluble in acid or basic medium, for example in the form of hard or soft gelatine capsules.

A use of the preparation according to the invention is provided for the controllable release of active substance in the gastric juice region. However, it may also be provided for the controllable release of active substance in the gastrointestinal tract, especially in the small intestine. Such difference depends, in a manner known per se, on the pH value of the body fluid in the acid region of the stomach on the one hand, or on the other in the neutral or basic region of the small intestine. Preferably the preparation serves to attain a freely modulatable control and especially a linear control of the release of active substance. Finally, the release of active substance may also be provided for in the large intestine.

Finally, the preparation may be utilized for the controllable release of active agent and auxiliary agent, for instance in the form of a moulded article such as a suppository in the anal and vaginal region, or as an implant.

Suitable active agents are found in the active substance groups of the parasympatholytics (e.g. scopolamine, atropine, berlactyzine), the cholinergics (e.g. physostigmine, nicotine), the neuroleptics (e.g. chlorpromazine, haloperidol), the monoamine oxidase inhibitors (e.g tranylcypromine, selegiline), the sympathomimetics (e.g ephedrine, D-norpseudoephedrine, salbutamol, fenfluramine), the sympatholytics and anti-sympathotonics (e.g. propanol, timolol, bupranolol, clonidin, dihydroergotamine, naphazoline), the anxiolytics (e.g. diazepam, triazolam), the local anaesthetics (e.g. lidocain), the central analgesics (e.g. fentanyl, sufentanil), the antirheumatics (e.g. indomethacin, piroxicam, lornoxicam), the coronary therapeutics (e.g. glycerol trinitrate, isosorbide dinitrate), the estrogens, gestagens and androgens, the antihistaminics (e.g. diphenhydramine, clemastin, terfenadine), the prostaglandin derivatives, the vitamins (e.g. vitamin E, cholecalciferol), the antitumor agents and the cardioactive glycosides such as, for instance, digitoxin and digoxin.

As components comprised in the base material of the layers containing active substance may be utilized polymers such as polyisobutylene, esters of polyvinyl alcohol, polyacrylic, polymethacrylic and polymethyl-methacrylic acid and their derivatives, natural rubber, styrene, isoprene and styrene-butadiene polymerisates or silicone polymers, resin components such as saturated and unsaturated hydrocarbon resins, derivatives of abietyl alcohol and β-pinene, softeners such as phthalic acid ester, triglycerides and fatty acids, as well as a number of further substances known to those skilled in the art.

For the layers configured as insoluble, a plurality of materials are in principle suitable, especially those acceptable for pharmaceutical products: polyvinyl alcohol, styrene-diene block copolymers, polyurethanes, polyvinyl chloride, polymethacrylates, polyacrylate, polymethyl acrylate, polymethyl methacrylate and derivatives, polyolefin as well as polyester, to mention but a few examples.

A process for manufacturing a preparation according to the invention is characterized by the steps listed in claim 20. One embodiment of the process provides that to achieve a desired release program after application parts of the active and/or auxiliary substance layer in the longitudinal extension of the laminate are removed or added. Also, further active layers may be laminated to the laminate. Finally, an ultimate step of the process provides for the preparation to be embedded in a substrate.

Further details, features and advantages of the invention will become apparent from the following illustration of some embodiment examples schematically represented in the drawings.

FIGS. Ia/b, FIGS. IIa-g, FIGS. III and IV, FIGS. Va/b as well as FIGS. VIa/b show, in side view or in plan view, preparations according to the invention in a substrate containing these preparations.

The embodiment according to FIG. Ia shows a pressure-sensitive adhesive water-soluble active substance layer (2) on the inside of the winding, with the active substance-free carrier layer (1) preventing a premature release of active agent.

When this rolled-up preparation is exposed to body fluids, the active substance-containing adhesive dissolves and partially unrolls the systems, during which process active substance can enter, by diffusion or solution, from the layer (2) into the body fluid in correspondence with the surface area which has been disposed at a given moment. The release profile is thus controlled by the geometry of the active substance layer, the slow unrolling of the system successively exposing new active substance-containing surfaces, and the release profile resulting from the layer geometry and the speed of unrolling.

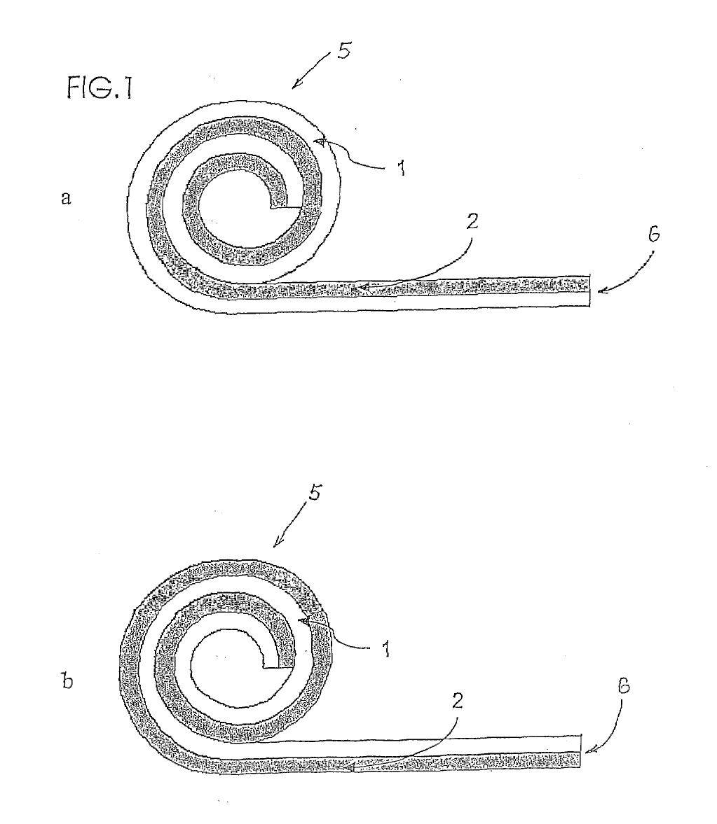

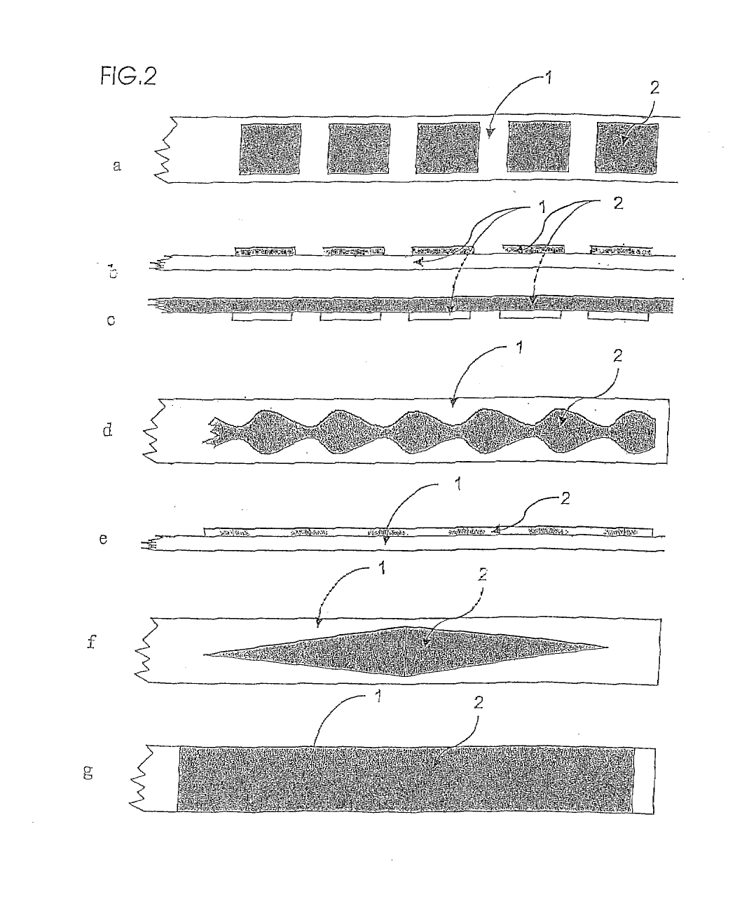

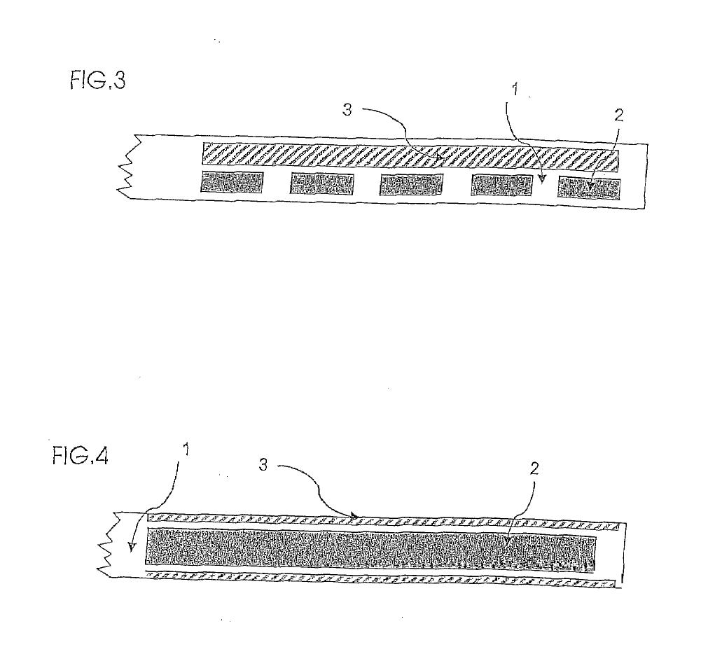

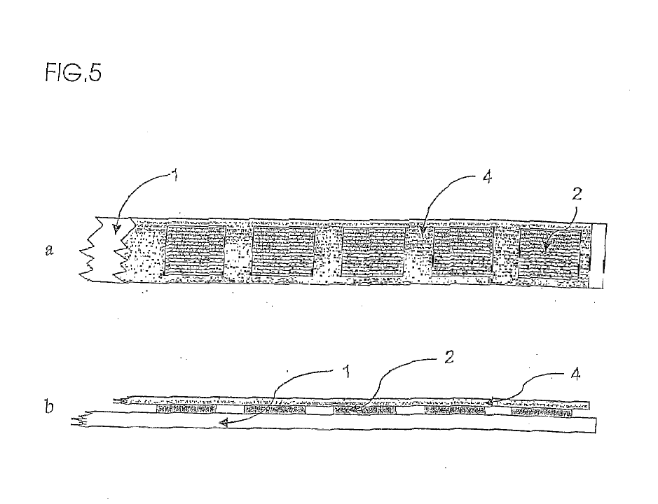

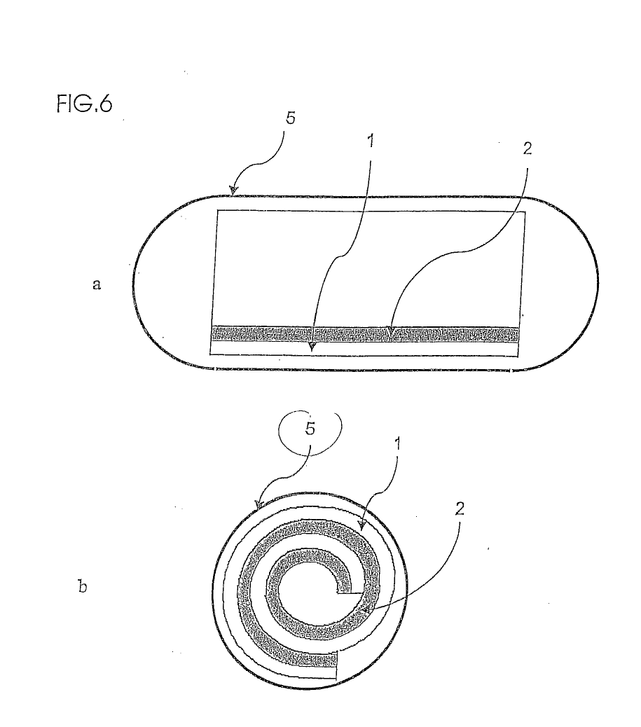

According to FIG. 1, the active substance-containing layer (2) is positioned on the outer side of the spiral whereas the inner winding is formed by the carrier layer. The advantage of this embodiment is the initial dose provided by the active agent-containing outer winding.

FIGS. IIa, d, f, g, each show different embodiments of the partially unrolled system in plan view, while FIGS. IId, c, e show the embodiments in side view. The system according to FIGS. IIa, b enables a temporally pulsed active substance release, while embodiments IId, e result in a modulated swelling or deflation of the release.

FIG. IIf relates to an embodiment providing a slow rise or drop in active substance. FIG. IIg shows a different embodiment providing a constant active substance release, as known in pharmaceutics as a release of zero order.

The invention moreover permits the release of different active substances with different release kinetics. For example, FIG. III shows an embodiment of this kind with the active substance layer being formed by two different regions (FIGS. III, 2, 3) carrying active substances; region 2 in the instant case providing a pulsed release whereas region 3 enables a continuous release of active agent.

The embodiment in FIG. IV also comprises two regions: Region 2 is configured as active substance-containing, water-soluble adhesive, region 3, by contrast, as largely active substance-free or active substance-containing, but water-insoluble sealing region. The water-insoluble sealing region at the edges has a protective function since without this barrier active substance would, in the unrolled state, be prematurely released via the sides. The stability of the rolled-up preparation in this case is produced by the centrally applied adhesive in region 2, and only insignificantly by the barrier region. Here, it is ensured that the active substance is released exclusively via the unrolled, exposed areas. However, it is also feasible that for this purpose the end faces of a rolled-up system are adhesively bonded or sealed such that the bond or seal is slowly soluble.

Furthermore, it may be of advantage to include a further layer in the system, which for example takes over the pressure-sensitive adhesive properties.

FIGS. Va, d shows an example of such an embodiment, in plan and side view, respectively. The said layer may be adapted so as to contain active agent or be free of active agent, and may be soluble or insoluble in water.

FIGS. VIa, b shows in plan and side view, respectively, the preparation of the invention in a substrate 5 containing said preparation, the substrate consisting of a substance soluble in acidic and/or basic medium. Such configuration of the invention may be advantageous if the carrier layer is erodible or soluble in water. Moreover, the pressure-sensitive adhesive layer can be insoluble.

FIG. VI shows the substrate 5 enveloping the preparation of the invention in plan view (VIa) and in side view (VIb), respectively. The preparation can be configured as hard or soft gelatine capsule, but may also be present in form of a suppository.

The invention can be realized in an uncomplicated manner and represents an optimal solution to the task posed at the outset.

1. A preparation containing active and/or auxiliary substance(s), for the time- and/or dose-controllable release of said substances, comprising a laminate made up of at least a carrier layer (1) and a matrix layer (2), said laminate being in rolled or folded shape, wherein
a) the matrix layer (2) has a longitudinal extension, contains at least one active or auxiliary substance, and is continuous at least in sections thereof,
b) at least one of the parameters of width and concentration of the active and/or auxiliary substance of this layer is not constant in relation to said longitudinal extension,
c) said carrier layer (1) is continuous and possesses a lower moisture permeability than the matrix layer (2), and
wherein at least one of said layers (1, 2) comprises a liquid-soluble adhesive which dissolves when the preparation is exposed to a body fluid.

2. The preparation according to claim 1, wherein in the longitudinal direction of the carrier layer (1), active substance-containing regions of the matrix layer (2) alternate at distances with active substance-free regions of the carrier layer (1).

3. The preparation according to claim 1, that further comprises at least one continuous and substantially moisture-impermeable layer.

4. The preparation according to claim 3, wherein the substantially moisture-impermeable layer contains one or more active substances and/or auxiliary substances.

5. The preparation according to claim 1, wherein the matrix layer (2) of the laminate is soluble or erodible in body fluid, and the carrier layer (1) is less readily soluble or more difficult to erode, or is insoluble or not erodable.

6. The preparation according to claim 1, wherein the concentration of the active substance or of the active substances varies in respect to the longitudinal extension of the active substance-containing layer(s), or is in the form of a concentration gradient or an otherwise variable concentration profile.

7. The preparation according to claim 1, wherein at least one layer is a pressure-sensitive adhesive layer.

8. The preparation according to claim 1, wherein the laminate is spirally rolled up, and said matrix layer (2) forms an outer layer of the spirally rolled-up laminate and contains active and/or auxiliary substances.

9. The preparation according to claim 1, wherein the laminate is spirally rolled up, and said matrix layer (2) forms an inner layer of the spirally rolled-up laminate and contains active and/or auxiliary substances.

10. The preparation according to claim 1, wherein one layer has regions with active and/or auxiliary substances, which regions differ in terms of their solubility, adhesive power or erosion properties.

11. The preparation according to claim 1, configured in form of a winding, that comprises a winding core which comprises a material which is soluble in body fluid.

12. The preparation according to claim 1, wherein in the center of the winding there is formed a tube recess of at least 0.5 mm in diameter.

13. The preparation according to claim 1, wherein the preparation effects a linear release of active substance.

14. The preparation according to claim 1, wherein the preparation effects the release of an initial dose.

15. The preparation according to claim 1, wherein those sides of a spirally rolled-up or folded preparation which correspond to longitudinal sides of the respective layers are provided with additional cover layers, said cover layers preferably containing substantially moisture-impermeable materials.

16. The preparation according to claim 1, wherein the preparation is embedded in a substrate (5) which comprises a substance that is soluble in an acidic or basic environment.

17. A method for the controlled release of an active and/or auxiliary substance in the anal or vaginal region, or as an implant, comprising:
administering the preparation according to claim 1 to said vaginal or anal region; or
implanting said preparation into the body.

18. A method for releasing active and/or auxiliary substances in the gastrointestinal tract, in the small intestine or in the large intestine, comprising:
administering the preparation according to claim 1 by oral application.

19. A method for releasing an active and/or auxiliary substance in the region of the gastric juice, comprising:
administering the preparation according to claim 1 by oral application.

20. A process of manufacturing the preparation according to claim 1, comprising:
providing a carrier layer (1),
coating said carrier layer (1) with at least one matrix layer (2) containing active and/or auxiliary substance, thus forming a laminate having a longitudinal extension,
drying of the laminate,
applying along the longitudinal extension of the laminate a thickness and/or width profile which can be modulated as required for achieving predeterminable release kinetics,
forming an application form from the preparation by rolling or folding, and
final packaging, wherein at least one of said layers (1, 2) comprises a liquid-soluble adhesive which dissolves when the preparation is exposed to a body fluid.

21. The process according to claim 20, wherein to achieve a desired release schedule following administration parts of the matrix layer (2) are removed or added in the longitudinal extension of the laminate.

22. The process according to claim 20, wherein further active layers (3, 4) are laminated to the laminate.

23. The process according to claim 20, wherein the preparation is embedded in a substrate (5).

24. The preparation of claim 1, configured in form of a winding, that comprises a winding core which comprises a material which is insoluble in body fluid.

25. The preparation of claim 1, in which the geometry and/or concentration of the active substance layer varies in the longitudinal direction of the matrix such that the release of at least one active substance is temporally in a pulsatile manner, in a manner in which the release is repeatedly modulated to increase and then decrease, or in a manner that is a rise in the release followed by a fall in the release.
